(19) United States
(12) Patent Application Publication
(10) Pub. No.: US 2006/0150970 A1
(43) Pub. Date: Jul. 13, 2006

(54) Apparatus and methods to titrate O2 content delivered to open delivery systems and mitigate fire risk

(75) Inventors: Samsun R. Lampotang, Gainesville, FL (US); Nikolaus L. Gravenstein, Gainesville, FL (US)

Correspondence Address: AKERMAN SENTERFITT, P.O. BOX 3188, WEST PALM BEACH, FL, 33402-3188, US

(73) Assignee: UNIVERSITY OF FLORIDA RESEARCH FOUNDATION, INC., Gainesville, FL (US)

(21) Appl. No.: 11/182,706

(22) Filed: Jul. 15, 2005

Related U.S. Application Data
(60) Provisional application No. 60/588,059, filed on Jul. 15, 2004.

(51) Int. Cl. A61M 15/00; A61M 15/08

(52) U.S. Cl. 128/203.12; 128/203.14; 128/203.22

(57) ABSTRACT

An apparatus and method for titrating the oxygen content delivered to a patient via an open delivery system and reduce fire risk. The apparatus may include a nasal cannula or face mask and an anesthesia machine having an air flowmeter. The cannula may be in fluid connection with one or more gas outlets on the anesthesia machine. The cannula may provide a user-adjustable reduced O₂ content gas flow to the patient. By providing an O₂ gas mixture having a lower concentration of O₂, the present invention significantly mitigates the risk of surgical fires. The apparatus may also include an anesthesia machine having an electronic flowmeter, either alone or in conjunction with the cannula, for maintaining oxygen saturation at a selected level. The apparatus may also include an anesthesia machine having an additional operating mode for use with an open delivery system.

### CROSS-REFERENCE TO RELATED APPLICATIONS

This application claims the benefit of and incorporates by reference in its entirety Provisional Patent application No. 60/588,059 filed on Jul. 15, 2004.

### STATEMENT REGARDING FEDERALLY SPONSORED RESEARCH OR DEVELOPMENT

Not applicable.

### FIELD OF THE INVENTION

The invention relates to medical devices. More specifically, the invention relates to apparatus and methods for delivering an O₂-containing gas mixture by nasal cannula or open delivery systems.

### BACKGROUND

Nasal cannula assemblies and facemasks have found widespread use to provide supplemental O₂ or other gases to a patient for use over a relatively long period of time. Nasal cannulae have largely replaced O₂ masks and provide much greater comfort than nasal catheters. The use of such devices has proved sufficiently beneficial that they are widely used not only by respiratory patients, but also for a wide variety of patients who benefit from the O₂ added by such assemblies.

The most commonly used arrangement includes a dual prong nose piece which is centered in a loop of vinyl tubing. The nose piece openings are inserted in the nose with the tubing tucked behind the ears.

Pure O₂ is generally delivered to patients via nasal cannulae. However, such a practice may pose fire risks. The Joint Commission on Accreditation of Healthcare Organizations (JCAHO) has issued a sentinel event alert about operating room fires and has recommended the use as a general policy and consistent with patient needs of about 30% O₂ or less when supplying supplemental O₂ via nasal cannula or other open delivery systems, such as face masks or face tents.

It is estimated that 50 to 100 surgical fires occur in the United States every year. The presence of an O₂-enriched atmosphere has been estimated to be involved in about 74% of all surgical fires.

Three elements are required for the classic fire triad: an ignition source (e.g., a spark), an oxidizer (e.g., free-flowing O₂), and fuel (e.g., bedding, drapes, patient hair, patient cap, patient clothing, skin prep solution, or sponges). A fire may occur when these three elements are present under the right conditions. Keeping these elements apart helps prevent surgical fires and protects the patient from injury, but separation of the various fire triad components is not always possible, practical or fully effective.

When an anesthesia machine is used to supply an open delivery system with a sub-100% O₂ gas mixture out of concerns for fire hazards, there are several constraints. The standard tapered inlet for a nasal cannula or face mask does not fit any of the outlet ports on the anesthesia machine except for the barbed outlet of an auxiliary O₂ flowmeter that delivers 100% O₂ and is thus contra-indicated per recent JCAHO recommendations.

### SUMMARY

The present invention provides an apparatus for delivering an O₂-containing gas mixture by an open delivery system such as a nasal cannula or face mask to a patient. The apparatus may include an anesthesia machine having an air flowmeter. The nasal cannula or face mask may be in fluid connection with one or more outlets on the anesthesia machine. The nasal cannula or facemask may include a fluid conduit terminating at a pair of apertured nostril outlet prongs or facemask, respectively, for providing the reduced O₂ content gas flow to the patient. By providing an O₂ gas mixture having a lower concentration of O₂, the present invention reduces the risk of surgical fires.

In particular, in one aspect, the present invention provides an apparatus for reducing a risk of fire using an anesthesia machine including an anesthesia machine having an air flowmeter and at least one blended-gas outlet; and a nasal cannula constructed and arranged to connect to the at least one blended-gas outlet.

In another aspect, the present invention provides an apparatus for reducing a risk of fire using an anesthesia machine including an anesthesia machine having an air flowmeter and at least one blended-gas outlet; a sensor for measuring blood oxygen saturation; and a control system for adjusting an O₂ concentration of blended gas supplied to a patient, a flowrate of blended gas supplied to a patient, or both.

In another aspect, the present invention provides an apparatus for reducing a risk of fire using an anesthesia machine including an anesthesia machine having an air flowmeter and at least one blended-gas outlet; and an operating mode selector wherein the selector is constructed and arranged such that when the machine is used for open delivery of blended gas, the selector is capable of adjusting at least one operating parameter selected from automatically closing any adjustable pressure limiting valves; resetting any sustained pressure alarms; readjusting any alarms for O₂ concentration in the blended gas; and a combination thereof.

### BRIEF DESCRIPTION OF THE DRAWINGS

Other objects, features and advantages of the invention will become apparent upon reading the following detailed description, while referring to the attached drawings, in which:

FIG. 1 shows a standard nasal cannula inlet.

FIGS. 2A-E show various embodiments of a nasal cannula inlet according to various embodiments of the present invention.

FIG. 3 provides a logic flow diagram for determining which embodiment of the present invention to use based upon various factors.

FIG. 4 provides a logic flow diagram for those embodiments using an anesthesia machine having software-controlled flowmeters.

FIG. 5 is a perspective view of a manufactured prototype of one embodiment of the present invention with an attached gas sampling line.

FIG. 6 is a close-up view of an inlet connector of a manufactured prototype.

FIG. 7 is a perspective view of the inlet connector of a manufactured prototype connected to the common gas outlet of an anesthesia machine.

FIG. 8 is a perspective view of the inlet connector of a manufactured prototype connected to the Y-piece of a breathing circuit

### DETAILED DESCRIPTION

The present invention is more particularly described in the following description and examples that are intended to be illustrative only since numerous modifications and variations therein will be apparent to those skilled in the art. As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of.”

The invention provides an apparatus and method for delivering an O₂-containing gas and/or reducing the risk of fire. In a preferred embodiment, the apparatus and method provides the capability to analyze the composition of the gas delivered to an open delivery system. The apparatus may also include a machine (not necessarily an anesthesia machine) having software controlled flowmeters, either alone or in conjunction with an open delivery system, for maintaining oxygen saturation at a selected level by modulating O₂ delivery, e.g., by altering the fraction of delivered oxygen (F_{D}O₂) and/or total delivered flowrate. The apparatus may also include an anesthesia machine having an additional operating mode for use with open delivery systems where alarm algorithms, thresholds and logic are automatically re-configured to avoid unnecessary and annoying alarms when supplying an open delivery system with an anesthesia machine.

The invention addresses a weakness in current design of open delivery systems by adding the capability for reducing the concentration of O₂ in gas supplied to a patient when supplying supplemental O₂ by nasal cannula and other open delivery systems. The present invention may also permit, in various embodiments, routine gas analysis of the gas composition delivered to an open delivery system. Gas analysis of delivered gas composition helps clinicians determine (a) if the set composition is above a threshold for increased fire hazard, (b) determine if the open delivery system has been accidentally disconnected from its gas supply and/or (c) whether the gas issuing from the oxygen source is actually oxygen.

The present invention, in one embodiment, provides a nasal cannula with a connector permits mating to a common gas outlet (CGO), an auxiliary common gas outlet (ACGO) and/or a Y-piece or other component of a breathing circuit, such as the inspiratory/expiratory ports or bag mount. In alternative embodiments, the nasal cannula may include gas sampling ports or lines that allow monitoring of the delivered gas concentration.

In another embodiment, the present invention provides an anesthesia machine having electronic flowmeters wherein the O₂ concentration delivered to an open delivery system may be modulated in response to blood oxygen saturation, such as the arterial oxygen saturation obtained from pulse oximetry (SpO₂). As such, this embodiment provides a closed loop system that is capable of optimizing oxygen content by enriching the O₂% and/or increasing total flow delivered in response to changes in SpO₂. As such, in these embodiments, the present invention is capable of making changes in delivered O₂ content to maintain SpO₂ at a set level or range while minimizing fire hazards. This embodiment may also employ the nasal cannulae of the present invention. In general, a total flow of 4 L/min is considered the threshold beyond which patient discomfort may ensue. Thus, F_{D}O₂ may be preferentially increased first before increasing total flow as a means to increase O₂ delivery.

In yet another embodiment, the present invention provides an anesthesia machine that includes an additional operating mode wherein certain alarms that are used for other operating modes are deactivated and/or reset to other limits or default values. As such, this embodiment enables an anesthesia machine to be operated with an open delivery system without the constant need to address nuisance alarms that unnecessarily sound. These alarms may distract and/or annoy the user and/or force the user to spend time responding to the alarms rather than on the procedure itself. This embodiment may be used in conjunction, but not necessarily, with software controlled flowmeters. The alarms are used to ensure proper O₂ delivery is occurring and/or there is no increase in risk of fire due to O₂ levels that are too high. These embodiments may also utilize the novel cannulae of the present invention.

As such, the present invention provides several techniques for lowering the resulting delivered O₂% to the patient. These techniques, which are provided in greater detail below, may generally be used either alone or in combination with the other techniques presented below.

More specifically, in one aspect, the present invention provides a nasal cannula for use with anesthesia machines. As shown in FIG. 1, a prior art cannula includes a tapered inlet 105 that leads to a cannula tubing 110 that channels the gas to the patient. The cannula 100 includes the tapered inlet 105 that may be easily connected to the barbed outlet of an auxiliary ball-in-tube O₂ flowmeter. However, the auxiliary ball-in-tube O₂ flowmeter generally found on anesthesia machines delivers 100% O₂. Pure O₂ delivered by cannula may create a localized, oxygen-rich environment that promotes surgical fires when combined with an ignition source and combustible material.

As such, the present invention provides various embodiments of cannulae that are capable of being used in conjunction with various outlets of an anesthesia machine that deliver air and oxygen in a blend that results in an O₂-containing gas having reduced concentrations of oxygen, and therefore, a reduced risk of fire. Prior art cannulae are not capable of being attached to these blended gas outlets and, as such, cannot reduce the risk of fire absent additional operating parameters. The reduced risk of fire is accomplished by using a cannula that is constructed and arranged to be attached to one or more outlets of an anesthesia machine that delivers a gas blend having a user-controllable fraction of delivered O₂ (F_{D}O₂), usually ≦30%, depending on the individual anesthesia machine configuration and the clinical logistics. For example, the F_{D}O₂ can be <25%, <20% or less than 15%. Additionally, in alternative embodiments, the F_{D}O₂ may be higher than 30%, such as 35%.

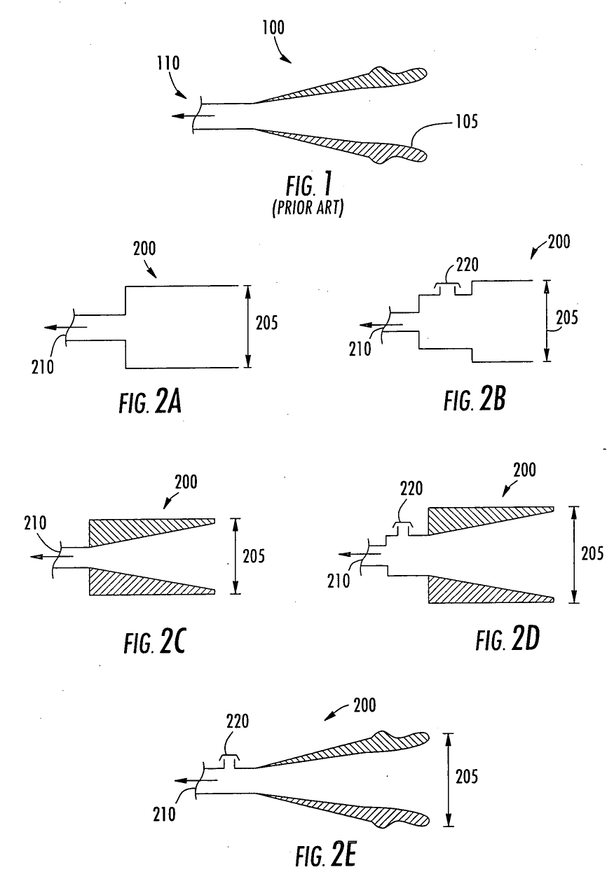

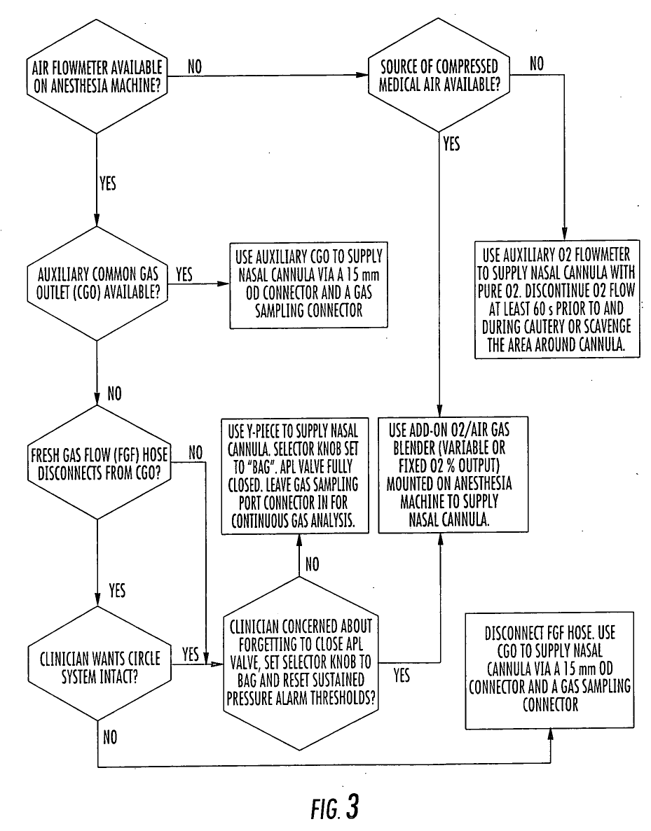

As seen in FIGS. 2A-2E, these various embodiments according to the invention utilize a connector 200 having an outer diameter 205 that is sized and shaped to be able to be connected to the Y-piece of an anesthesia machine, the CGO of an anesthesia machine, and/or the ACGO of an anesthesia machine, as well as possible other ports or bag mounts. In alternative embodiments, the connector may be designed such that it cannot be connected to certain barbed inlets, such as those commonly associated with an auxiliary O₂ flowmeter that delivers 100% O₂. As such, the connector would not be capable of being in a manner that may deliver gas having a much higher concentration of O₂ than beneficial. As can be seen, the inlet connector 200 may include tapered inlets (FIGS. 2C-2E) that fit a barbed outlet, or inlet configurations deliberately designed to be incompatible with a barbed outlet such as a cylindrical inlet (FIGS. 2A-2B). It should be noted that, in some embodiments, the cannula inlet connector may have a very slight taper that would not be discernable to the naked eye. This slight taper may be used to ensure an airtight seal.

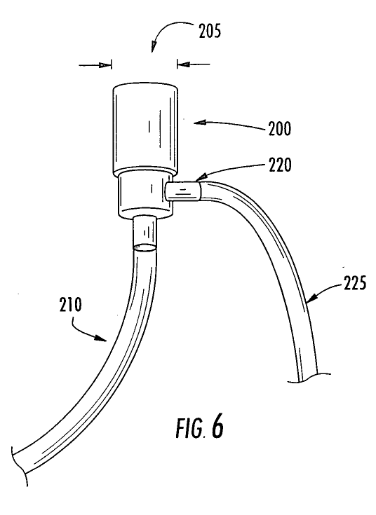

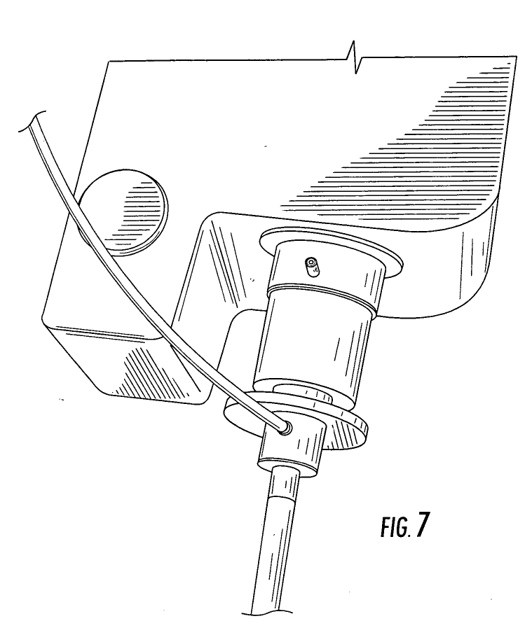

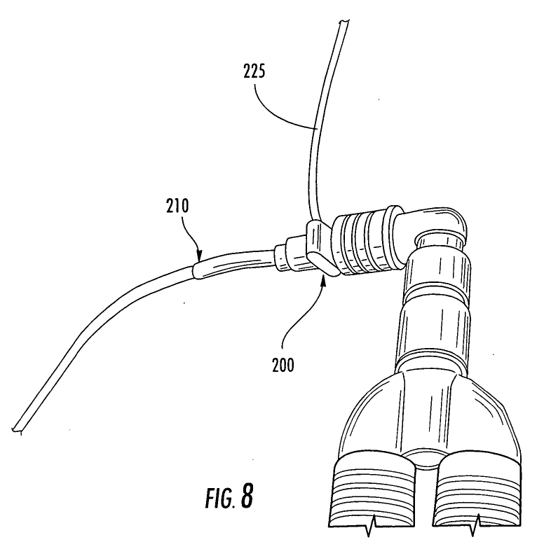

In alternative embodiments of the invention, an O₂ sensor or gas sampling port or line may be disposed in fluid communication with the nasal cannula. FIGS. 2B, 2D and 2E all show a nasal cannula with an inlet connector 200 having a gas sampling port 220. A gas sampling line (not shown in FIG. 2, 225 in FIGS. 5, 6 and 8) may be detachably connected to the gas sampling port 220. In the embodiment shown in FIG. 5, the gas sampling line may be permanently attached to the cannula inlet connector 200. The gas sampling line supplies an O₂ sensor (not shown) that may then be used to sample or measure the percentage of O₂ flowing to the patient through the cannula tubing 210. The sensor may generate an electrical output based on the measured percentage composition of O₂. The O₂ sensor may be located remote from inlet connector 200 and include a small pump (not shown) for aspirating a gas sample through gas sampling port 220 and gas sampling line 225. Alternatively the O₂ sensor may be mounted directly on cannula 200.

The O₂ sensor may be a fully integrated component in some anesthesia machines, an add-on unit to an anesthesia machine, a stand-alone unit or part of a multi-gas analyzer. The aspiration may be, but is not necessarily, continuous. The sensor output may then amplified, filtered and A/D converted by interface circuitry and then forwarded to a microprocessor-based controller. The controller can include a memory that stores an O₂ set point, which is the level that a clinician generally sets. The controller may then compare the output of the O₂ sensor to the set point level of O₂. The controller may then generate a response signal based on the comparison, which is communicated to a driver circuit.

The location of the gas sampling port 220 on the inlet connector 200 relative to the inlet plane and the flow restriction as diameter 205 narrows down to the cannula tubing 210 may predispose to gas analyzer alarms such as incorrect air leak messages. The location of the sampling port takes into consideration among others, minimizing gas analyzer alarms and ease of manufacturing. A beneficial location of the sampling point is downstream of the narrowing of the inlet connector. The pressure is less downstream of the constriction and the sampling port does not present mechanical interference that might prevent an airtight seal.

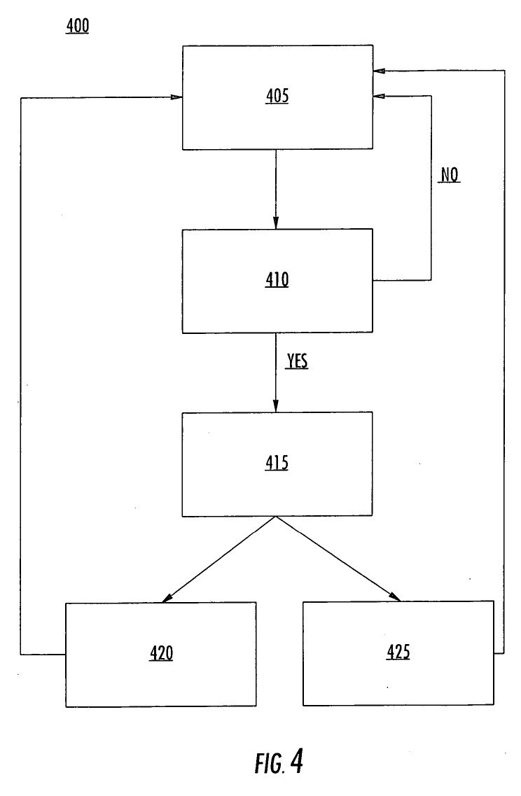

The nasal cannula of the present invention may be used as one potential technique for reducing F_{D}O₂ to an individual through use of this cannula with an anesthesia machine having an air flowmeter. As shown in FIG. 3, there are methods for reducing the risk of fire in those instances where the anesthesia machine does not have an air flowmeter. In anesthesia machines without an air flowmeter, one option is an add-on air/O₂ gas blender if there is a source of compressed air, although this option comes with added expense, plumbing, mounting hardware and clutter. If there is no source of compressed air, then the existing solution is to use 100% O₂ from the auxiliary O₂ flowmeter and discontinue oxygen supplementation for at least 60 seconds prior to and during cautery use, and/or empirically scavenge in the region of the face, head and neck.

However, when the anesthesia machine includes an air flowmeter, then the cannula of the present invention may be used and may be connected to various blended-gas outlets based upon the manner and/or need in which the anesthesia machine will be used. As the cannula is connected to a blended gas outlet, and not 100% pure O₂, this results in the reduced risk of fire.

Anesthesia machines with an air flowmeter are capable of blending a ≦30% O₂ mixture in air and delivering it to the CGO and, if present, also the ACGO. A nasal cannula of the present invention may be mated directly, to the CGO, a Y-piece (via the CGO) or, if present, the ACGO. To obtain an F_{D}O₂ ≦30%, the oxygen-to-air ratios may be adjusted to such that they are equal to or less than about 1:7 (e.g. 0.5 L/min O₂: 3.5 L/min air or 1 L/min O₂: 7 L/min air). As shown in FIG. 3, in those anesthesia machines having an ACGO, the blended gas mixture may be rerouted to either the CGO or the ACGO by adjustment of a selector lever.

If the anesthesia machine has an ACGO, then the ACGO may be used to supply the gas to the patient using a cannula of the present invention. If no ACGO is available, then a determination is made whether the fresh gas flow (FGF) hose can be disconnected from the CGO. If not, or if it can be disconnected but the clinician wants the circle system to remain intact, then a determination is made whether the clinician is concerned about forgetting to close the adjustable pressure limiting (APL) valve, and/or setting the selector knob to “bag” and resetting the sustained pressure alarms. If not, then the Y piece may be used to supply the nasal cannula. In this embodiment, the selector knob is set to “bag” and the APL valve is fully closed.

If the clinician is concerned about forgetting to close the APL valve, and/or setting the selector knob to “bag” and resetting the sustained pressure alarms, then the add-on air/O₂ gas blender may be used, but with the corresponding added expense, plumbing, mounting hardware and clutter.

However, according to one embodiment of the present invention, if the FGF hose may be disconnected, and the clinician does not want the circle system to remain intact, then the FGF hose may be disconnected and the CGO may be used to supply the nasal cannula.

Accordingly, as can be seen from FIG. 3, the nasal cannula of the present invention may, in various embodiments, be used to supply an O₂-containing gas to a patient using an anesthesia device having an air flowmeter. Additionally, the cannula may be connected to the CGO, ACGO or Y connector, depending on a variety of factors.

In another technique for reducing the risk of fire according to the present invention, the present invention also provides the capability to measure the degree of blood oxygen saturation and use this measurement to control the concentration of the O₂ in the gas delivered to the patient and/or the level of gas flow delivered to the patient.

In this embodiment, the anesthesia machine may utilize a software-controlled or electronic flowmeter (such as the Datex Ohmeda Avance) wherein the O₂ concentration in the gas delivered to an open delivery system may be increased independently of the total flow of the gas delivered. These changes in concentration and/or flow may be made in response to oxygen saturation as determined by pulse oximetry (SpO₂). As such, the present invention provides a closed loop system that optimizes oxygen delivery, e.g., by altering the O₂% and/or modifying total flow delivered in response to increases and/or decreases in SpO₂ and that has the ability to make changes in delivered O₂ content to maintain SpO₂ at a set level or range while minimizing fire hazards.

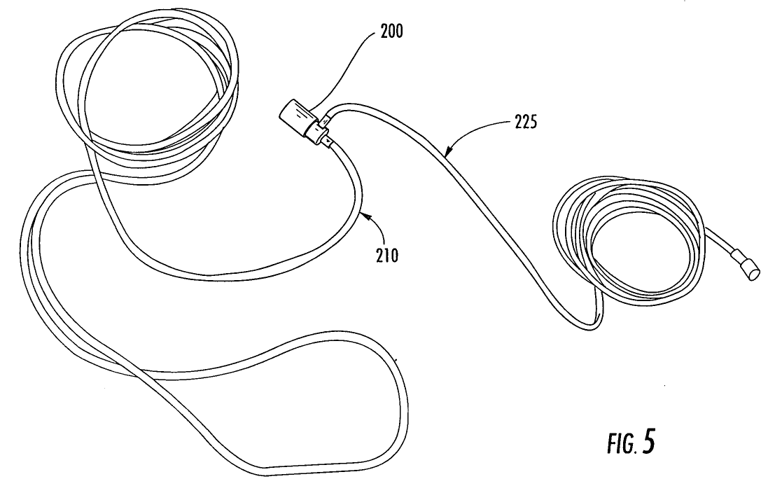

More particularly, and referring to FIG. 4, the present invention provides a system 400 for maintaining or optimizing oxygen saturation in the blood while reducing the risk of fire hazards. In the system 400, a blood oxygen sensor such as a pulse oximeter 405 is used to measure blood oxygen saturation in a patient. Then, in step 410, the measured value is compared to a preset value or a preset range of values. If the value falls within the present range, then the system returns to step 405. If the value falls outside the selected range, then, in step 415, the system adjusts the delivery of O₂ to achieve the selected saturation. The pulse oximeter may be a fully integrated component in some anesthesia machines, an add-on unit to an anesthesia machine, a stand-alone unit or part of a multi-parameter physiological monitor.

The system 400 may adjust the delivery of O₂ by changing the concentration of the O₂ in the gas mixture, the total gas flow rate, or both. In step 420, the system may examine the concentration of O₂ in the gas. An initial upper limit of 30% O₂ may be selected as a control to help reduce the risk of fire. If the concentration of the O₂ in the gas is less than 30%, and an increase in blood oxygen saturation is needed, then the system may increase the concentration of the O₂ in the gas until this concentration reaches 30%. After a period to permit the increase to be reflected in blood oxygen saturation, the system returns to step 405 and blood oxygen saturation is retested. If additional changes are needed, then the system proceeds as before.

As an alternative, if the concentration of the O₂ in the gas is 30%, or if the system does not select this alternative, then, in step 425, the system may increase the gas flow rate. An initial upper limit of 4 L/min may be used, as higher flow rates may tend to be uncomfortable to the patient and/or causing drying of the nasal passages. If the flow rate of the gas is less than 4 L/min, and an increase in blood oxygen saturation is needed, then the system may increase the flow rate until this reaches 4 L/min. Again, after a period to permit the increase to be reflected in blood oxygen saturation, the system returns to step 405 and blood oxygen saturation is retested. If additional changes are needed, then the system proceeds as before.

In another embodiment, the concentration of the O₂ in the gas and the flow rate may both be adjusted, such that the system performs both steps 420 and 425. Also, in alternative embodiments, the initial upper limits of 30% and 4 L/min may be changed if these limits have been reached and blood oxygen saturation is still below the selected level. The system 400 may also be programmed such that the concentration of the O₂ in the gas is raised first up to the limit before flow rate is adjusted, or that the flow rate is raised first up to the limit before the concentration of the O₂ in the gas is adjusted, or any alternative between, based upon parameters and factors that may be altered on a situational basis. Conversely, if the blood oxygen saturation is satisfactory and/or at or above the desired set point and the total flow rate and/or F_{D}O₂ exceed their respective set points, the system will reduce the total flowrate and F_{D}O₂ either individually or together to optimize blood oxygen saturation while mitigating fire risks. The system will allow clinicians to set the parameter, blood oxygen saturation or F_{D}O₂, that takes precedence in the control loop depending on the clinical context. For example, if there is much cautery going on and the patient is relatively healthy, F_{D}O₂ may be set as the dominant parameter with blood oxygen content subservient to it. Conversely, with a patient prone to desaturation and in an procedure where there is minimal, predictable or no cautery or other ignition sources, blood oxygen saturation may be set as the dominant parameter with F_{D}O₂ as a secondary optimization parameter.

In addition to the cannula and the method of controlling an anesthesia machine using blood oxygen saturation and possibly the cannula, the present invention also includes an anesthesia machine having an additional mode of operation that may be used with the cannula and that may be controlled by measuring blood oxygen saturation.

In this aspect, the present invention provides an additional mode of operation for an anesthesia machine. This additional mode may be labeled “open delivery,” although there is no requirement for the label other than to distinguish this mode from the other two modes.

If the cannula of the present invention is used with an anesthesia machine, there are certain issues that arise when operating the anesthesia machine in either “bag” or “ventilator” mode. If the cannula is used to deliver the gas mixture blended by the anesthesia machine and the machine is in “ventilator” mode, then the majority of the flow would likely escape through the ventilator pressure relief valve, instead of flowing to the open delivery system.

As such, it is beneficial for the cannula to be used in “bag” mode. In this embodiment, the user would set the selector knob to “bag” and close the Adjustable Pressure Limiting (APL) valve or else the majority of the flow would escape through the open APL valve, instead of flowing to the open delivery system. In designs with an auxiliary common gas outlet (ACGO), the user would have to remember to flip the lever that routes the blended gas mixture issuing from the flowmeter bank to the ACGO instead of the CGO. The user may also have to perform some mental math to individually set the air and O₂ flows to arrive at the desired total flow and O₂% in some machine designs not having an automatic controller.

One issue, though, is that the flow resistance of an open delivery system will usually be higher than that of the normal flow path, i.e., the fresh gas flow hose or its equivalent. Also flow is generally continuous such that, after equilibration, there will be a sustained airway pressure (typically of 20-25 cm H₂O) instead of the intermittent pressure cycling associated with ventilation that the machine expects to see while in “bag” or “ventilator” mode. Thus sustained pressure alarms and apnea alarms may unnecessarily sound when using an anesthesia machine to supply an open delivery system, providing nuisance alarms that distract and annoy the user and requiring the user to spend the time to figure out how to disable or adjust the alarm settings to eliminate the alarms. In addition, if the user successfully modifies the alarm thresholds to eliminate the alarms while supplying an open delivery system from an anesthesia machine, the user has to remember to reset the alarm thresholds back to the default settings for the next case.

Accordingly, by using an anesthesia machine having an open delivery mode of operation, and selecting this mode, these alarms and defaults may be deactivated or reset (optionally according to the total flow rate delivered to an open delivery system) to compensate for the back pressure imposed by the constant flow through, for example, the nasal cannula of the present invention. Depending on the anesthesia machine design, the “open delivery” mode may automatically place the machine in “bag” mode and close the APL valve. Upper and lower alarm limits for delivered O₂ fraction would be automatically set to say, for example, 30% and 22% respectively. The 30% alarm limit would sound to warn of an increased risk of a surgical fire. The 22% alarm limit would sound if there was a disconnection such that the cannula is no longer supplied with O₂ and the delivered fraction of O₂ becomes 21%, the O₂ concentration in ambient air.

In an alternative embodiment, the feedback system that optimizes delivered O₂ content on the basis of blood oxygen saturation or oxygenation status and fire hazard, as previously described, may also be part of the “open delivery” mode of the anesthesia machine. The “open delivery” mode, in another alternative embodiment, may also automatically route the fresh gas flow to the ACGO and automatically set default O₂% and total FGF level in some machine designs.

Although the invention will have significant utility in the operating room, the invention is in no way limited to use in the operating room. The invention may be used more-generally for procedures where some type of cautery is in use and/or less than 100% O₂ fulfills applicable clinical and safety considerations.

It is to be understood that while the invention has been described in conjunction with the specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

We claim:

1. An apparatus for reducing a risk of fire using anesthesia machines, comprising:
an anesthesia machine having an air flowmeter and at least one blended-gas outlet; and
an open delivery system selected from a nasal cannula or face mask connected to the at least one blended-gas outlet.

2. The apparatus of claim 1, wherein the at least one blended-gas outlet is selected from a common gas outlet, an auxiliary common gas outlet, a Y-piece, inspiratory port, expiratory port, or a combination thereof.

3. The apparatus of claim 1, wherein the at least one blended-gas outlet is capable of delivering a blended gas having an O₂ concentration of less than 100%.

4. The apparatus of claim 3, wherein the at least one blended-gas outlet is capable of delivering a blended gas having an O₂ concentration of less than or equal to about 30%.

5. The apparatus of claim 1, wherein the open delivery system further includes a gas sampling port.

6. The apparatus of claim 5, wherein the apparatus further includes an oxygen sensor for measuring oxygen concentration in fluid connection with the gas sampling port for measuring the oxygen concentration of blended gas supplied to the open delivery system.

7. The apparatus of claim 6, wherein the apparatus further includes a control system for adjusting an O₂ concentration of blended gas supplied to the open delivery system, a flowrate of blended gas supplied to the open delivery system, or both;
wherein the control system includes a pulse oximeter for measuring oxygen saturation of a patient's blood.

8. The apparatus of claim 1, wherein the anesthesia machine further includes an operating mode selector wherein the selector is constructed and arranged such that when the machine is used for delivery of blended gas to open delivery systems, the selector is capable of adjusting at least one operating parameter selected from automatically closing any adjustable pressure limiting valves; resetting any sustained pressure alarms; readjusting any alarms for O₂ concentration in the blended gas; rerouting the blended gas to an auxiliary common gas outlet; or a combination thereof.

9. An apparatus for reducing a risk of fire using an anesthesia machine comprising:
an anesthesia machine having an air flowmeter and at least one blended-gas outlet;
a sensor for measuring oxygen saturation; and
a control system for adjusting an O₂ concentration of blended gas supplied to a patient, a flowrate of blended gas supplied to a patient, or both.

10. The apparatus of claim 9, wherein the sensor for measuring oxygen saturation is a pulse oximeter.

11. The apparatus of claim 10, wherein the pulse oximeter is part of an anesthesia workstation.

12. The apparatus of claim 11, further comprising a nasal cannula or face mask connected to the at least one blended-gas outlet.

13. The apparatus of claim 12, wherein the at least one blended-gas outlet is selected from a common gas outlet, an auxiliary common gas outlet, a Y-piece, an inspiratory port, an expiratory port, or a combination thereof.

14. The apparatus of claim 12, wherein the nasal cannula is constructed and arranged such that the cannula is not capable of being connected to a barbed outlet of an auxiliary O₂ flowmeter.

15. The apparatus of claim 9, wherein the anesthesia machine further includes an operating mode selector wherein the selector is constructed and arranged such that when the machine is used for delivery of blended gas to open delivery systems, the selector is capable of adjusting at least one operating parameter selected from automatically closing any adjustable pressure limiting valves; resetting any sustained pressure alarms; readjusting any alarms for O₂ concentration in the blended gas; rerouting the blended gas to an auxiliary common gas outlet; or a combination thereof.

16. An apparatus for reducing a risk of fire using anesthesia machines, comprising:
an anesthesia machine having an air flowmeter and at least one blended-gas outlet; and
an operating mode selector wherein the selector is constructed and arranged such that when the machine is used for open delivery of blended gas, the selector is capable of adjusting at least one operating parameter selected from automatically closing any adjustable pressure limiting valves; resetting any sustained pressure alarms; readjusting any alarms for O₂ concentration in the blended gas; rerouting the blended gas to an auxiliary common gas outlet; or a combination thereof.

17. The apparatus of claim 16, further comprising a nasal cannula or face mask connected to the at least one blended-gas outlet.

18. The apparatus of claim 17, wherein the at least one blended-gas outlet is selected from a common gas outlet, an auxiliary common gas outlet, a Y-piece, an inspiratory port, an expiratory port, or a combination thereof.

19. The apparatus of claim 17, wherein the nasal cannula or face mask further includes a gas sampling port.

20. The apparatus of claim 19, wherein the apparatus further includes a sensor for measuring oxygen saturation, further wherein the sensor comprises a pulse oximeter.

21. The apparatus of claim 20, wherein the apparatus further includes a control system for adjusting an O₂ concentration of blended gas supplied to the cannula, a flowrate of blended gas supplied to the cannula, or both.
